(19) United States
(12) Patent Application Publication
(10) Pub. No.: US 2025/0269090 A1
(43) Pub. Date: Aug. 28, 2025

(54) MULTI-NETWORK HYDROGELS AS SYNTHETIC CARTILAGE

(71) Applicant: The Texas A&M University System, College Station, TX (US)

(72) Inventors: Melissa A. Grunlan, College Station, TX (US); Connor J. Demott, Bryan, TX (US)

(73) Assignee: The Texas A&M University System, College Station, TX (US)

(21) Appl. No.: 18/858,303

(22) PCT Filed: Apr. 18, 2023
(86) PCT No.: PCT/US2023/018912

§ 371 Date: Oct. 19, 2024

Related U.S. Application Data
(60) Provisional application No. 63/332,437, filed on Apr. 19, 2022.

(51) Int. Cl. A61L 27/52; A61L 27/54; A61L 27/56; C08J 3/075; C08J 3/24

(52) U.S. Cl. CPC A61L 27/52; A61L 27/54; A61L 27/56; C08J 3/075; C08J 3/246; A61L 2300/412; A61L 2300/606; A61L 2430/06; C08J 2333/26

(57) ABSTRACT

In an embodiment, the present disclosure pertains to a multi-network hydrogel composed of a first network, a second network, and a third network. In some embodiments, the first network, the second network, and the third network form a poly(2-acrylamido-2-methylpropane sulfonic acid) (PAMPS)/A-isopropylacrylamide (NIPAAm) copolymerized with acrylamide (AAm)/poly((3-acylamidopropyl)trimethylammonium chloride) (PAMPS/P (NIPAAm-co-AAm)/PAPTAC) triple network hydrogel. In another embodiment, the present disclosure pertains to synthetic cartilage compositions composed of the multi-network hydrogels as disclosed herein. In a further embodiment, the present disclosure pertains to methods of forming the multi-network hydrogels of the present disclosure.

### CROSS-REFERENCE TO RELATED APPLICATIONS

This patent application claims priority from, and incorporates by reference the entire disclosure of U.S. Provisional Patent Application No. 63/332,437 filed on Apr. 19, 2022.

### TECHNICAL FIELD

The present disclosure relates generally to synthetic cartilage and more particularly, but not by way of limitation, to multi-network hydrogels as synthetic cartilage.

### BACKGROUND

This section provides background information to facilitate a better understanding of the various aspects of the disclosure. It should be understood that the statements in this section of this document are to be read in this light, and not as admissions of prior art.

Repair of cartilaginous tissues has remained limited due to their low healing capacity and reliance on grafting procedures. Synthetic replacements, such as focal resurfacing devices and artificial intervertebral discs, have emerged but continue to suffer from mechanical mismatch and inadequate lubricity stemming from a lack of hydration.

### SUMMARY OF THE INVENTION

This summary is provided to introduce a selection of concepts that are further described below in the Detailed Description. This summary is not intended to identify key or essential features of the claimed subject matter, nor is it to be used as an aid in limiting the scope of the claimed subject matter.

In an embodiment, the present disclosure pertains to a multi-network hydrogel composed of a first network, a second network, and a third network. In some embodiments, the first network, the second network, and the third network form a poly(2-acrylamido-2-methylpropane sulfonic acid) (PAMPS)/N-isopropylacrylamide (NIPAAm) copolymerized with acrylamide (AAm)/poly((3-acylamidopropyl)trimethylammonium chloride) (PAMPS/P (NIPAAm-co-AAm)/PAPTAC) triple network hydrogel.

In another embodiment, the present disclosure pertains to synthetic cartilage compositions composed of the multi-network hydrogels as disclosed herein.

In a further embodiment, the present disclosure pertains to methods of forming the multi-network hydrogels of the present disclosure.

### BRIEF DESCRIPTION OF THE DRAWINGS

A more complete understanding of the subject matter of the present disclosure may be obtained by reference to the following Detailed Description when taken in conjunction with the accompanying Drawings wherein:

FIG. 1 illustrates DSC thermograms of TN-APTAC hydrogels, illustrating the volume phase transition temperature (VPTT) is tuned out of physiologic range—confirming dimensional stability under physiologic conditions.

FIGS. 2A-2C illustrate compressive mechanical properties of TN-APTAC hydrogels. FIG. 2A shows elastic modulus (compared to articular cartilage, TMJ disc, and IVDs). FIG. 2B shows strength (stress at fracture). FIG. 2C shows toughness. Note: *'s indicate statistical difference (p<0.05) from DN-AAm-10% and “ [symbol] ” indicates PEG-DA value; SN-AMPS-1.5M was not accounted for during statistical analysis.

FIGS. 3A-3C illustrate tensile mechanical properties of TN-APTAC hydrogels. FIG. 3A shows elastic modulus (compared to annulus fibrosus toe modulus and tracheal cartilage). FIG. 3B shows strength (stress at fracture). FIG. 3C shows toughness. Note: *'s indicate statistical difference (p<0.05) from DN-AAm-10%.

### DETAILED DESCRIPTION

It is to be understood that the following disclosure provides many different embodiments, or examples, for implementing different features of various embodiments. Specific examples of components and arrangements are described below to simplify the disclosure. These are, of course, merely examples and are not intended to be limiting. The section headings used herein are for organizational purposes and are not to be construed as limiting the subject matter described.

Repair of cartilaginous tissues has remained limited due to their low healing capacity and reliance on grafting procedures. Synthetic replacements, such as focal resurfacing devices and artificial intervertebral discs, have emerged but continue to suffer from mechanical mismatch and inadequate lubricity stemming from a lack of hydration. Multi-network hydrogels are a promising avenue to overcome these limitations by leveraging internetwork interactions. Previously, double network (DN) hydrogels based on poly(N-isopropylacrylamide) (PNIPAAm) double network (DN) hydrogels were developed that exhibited articular cartilage mimetic mechanical and hydration properties. However, the human body is composed of numerous forms of cartilage having higher moduli. Herein, triple network (TN) hydrogels are prepared to leverage hydrophobic associations and electrostatic internetwork interaction to achieve unprecedented moduli that align with high modulus cartilage types. These TN hydrogels are composed of an anionic poly(2-acrylamido-2-methylpropane sulfonic acid) (PAMPS) first network, a neutral N-isopropylacrylamide (NIPAAm) copolymerized with acrylamide (AAm) second network, and a cationic poly((3-acylamidopropyl)trimethylammonium chloride) (PAPTAC) third network (PAMPS/P (NIPAAm-co-AAm)/PAPTAC TN hydrogels). These hydrogels leverage internetwork physical crosslinks to increase the modulus (˜3 MPa) without sacrificing water content (˜80%). Correspondingly, this bolsters the capacity of hydrogels as a class of synthetic cartilage replacements, promoting use in applications requiring ultra-high moduli.

A myriad of cartilage types spans the human body, performing functions that necessitate high moduli (˜1-10 MPa, compressive). Table 1 below illustrates properties of cartilage throughout the human body (Ec=compressive modulus, ET=tensile modulus, E_{TC}=circumferential tensile modulus).

TABLE 1
Moduli of human cartilage

| | E_{T} | | E_{c} | E_{Tc} |
|---|---|---|---|---|
| Tracheal Cartilage | 2-15 | MPa | | |

| | | |
|---|---|---|
| Intervertebral Disc | 3-10 | MPa |

| | | |
|---|---|---|
| Annulus Fibrosus | 2.5 | MPa |

| Temporomandibular | 2 | MPa |
|---|---|---|
| Disc | | |
| Costal Cartilage | 5 | MPa |

| | | | | |
|---|---|---|---|---|
| Meniscus | 0.5 | MPa | 4-60 | MPa |

When these tissues are lost or damaged, repair is limited due to the low healing capacities associated with avascularity. Clinical repair strategies are primarily focused on auto- or allografting procedures, including osteochondral autograft transfer (OATS) for articular cartilage, and laryngotracheal reconstruction with harvested coastal cartilage. Nonetheless, these grafting treatments commonly suffer from donor site morbidity, graft availability, fibrocartilage formation, and graft resorption. To overcome these limitations, synthetic replacements are of significant interest; several have emerged (e.g., artificial intervertebral discs and articular cartilage focal resurfacing implants). The loading that must be endured has prompted the use of synthetic materials whose moduli greatly exceeds that of native cartilage types. However, this resulting mechanical mismatch is a primary source of failure, wherein altering mechanical stimuli of adjacent cartilage or bone leads to its deterioration. Moreover, modulus mismatch is associated with poor integration of the replacement material with surrounding native cartilage, arising from unequal strain responses to applied load. Joint functionality is also highly dependent on modulus matching (e.g., preservation of disc height while replicating motion segment mechanics). Native cartilage is characterized by significant water content as resulting osmotic forces give rise to their characteristic moduli and viscoelastic properties. Therefore, cartilage substitutes are ideally based on synthetic materials that not only achieve similar moduli, but also do so with similar hydration.

Hydrogels are a distinct class of materials associated with water contents that parallel cartilage tissues. However, the vast majority have moduli orders of magnitude lower than that of human cartilage types. For instance, poly(ethylene gycol)diacrylate (PEG-DA) hydrogels only reach a modulus of ˜200 KPa. Multi-network hydrogels have emerged as a unique class with exceptional mechanical properties. Double network (DN) hydrogels leveraging an anionic first network that obtain ultra-high strengths in the MPa range and high-water contents of ˜80% have been developed. Recent work has yielded a DN hydrogel that mimics the mechanical properties of articular cartilage, including a modulus in the MPa range. These hydrogels were composed of asymmetrically crosslinked networks of poly(2-acrylamido-2-methylpropane sulfonic acid) (PAMPS) and N-isopropylacrylamide (NIPAAm) copolymerized with acrylamide (AAm) (PAMPS/P [NIPAAm-co-AAm]). Superior mechanical properties of these hydrogels were afforded by the incorporation of PNIPAAm in the second network, which allowed for an apparent increase in crosslinking density through reversible hydrophobic interactions. This allowed for the DN hydrogels to reach strengths of ˜20 MPa and elastic moduli of ˜1 MPa while maintaining a cartilage like water content (˜84%). Further study of these PNIPAAm DN hydrogels illustrated their ability to mimic articular cartilage's tribological properties. In a synovial fluid simulant, mixed-boundary lubrication was suppressed and DN hydrogels were pushed to similar elasto-hydrodynamic regimes as articular cartilage. While these PNIPAAm DN hydrogels show promise towards serving as a resurfacing material for articular cartilage, their modulus does not extend into the range of other cartilaginous tissues (e.g., IVD or tracheal cartilage).

Inclusion of a third network has been shown to impact a variety of physical properties in hydrogels. This ranges from modulus, strength, and toughness, to lubrication properties. Triple network (TN) hydrogels were first introduced by adding an additional network of PAMPS to their original DN hydrogel (PAMPS/PAAm). These TN hydrogels attained an elastic modulus of ˜2 MPa and a coefficient of friction as low as 10-4. Uniquely, this was done without diminishing the strength of the hydrogel (4.8 MPa) or notably decreasing the water content (82.5%), compared to the DN hydrogel. Similarly, the inclusion of a known biomimetic boundary lubricant, poly(2-methacryloyloxyethyl phosphorylcholine), as a third network, produced a TN hydrogel capable of both biphasic and boundary lubrication. These TN hydrogels exhibited lower coefficients of friction across a wide range of sliding speeds than their DN hydrogel counterparts. Using an alternate approach, nonionic TN hydrogels were developed which had linear (i.e., non-crosslinked) second and third networks. These hydrogels exhibited increasing strengths with the addition of a third network, where the greatest increases were seen when using poly(N,N-dimethylacrylamide) for all three networks, which allowed for hydrophobic interactions across networks (i.e., physical internetwork crosslinks).

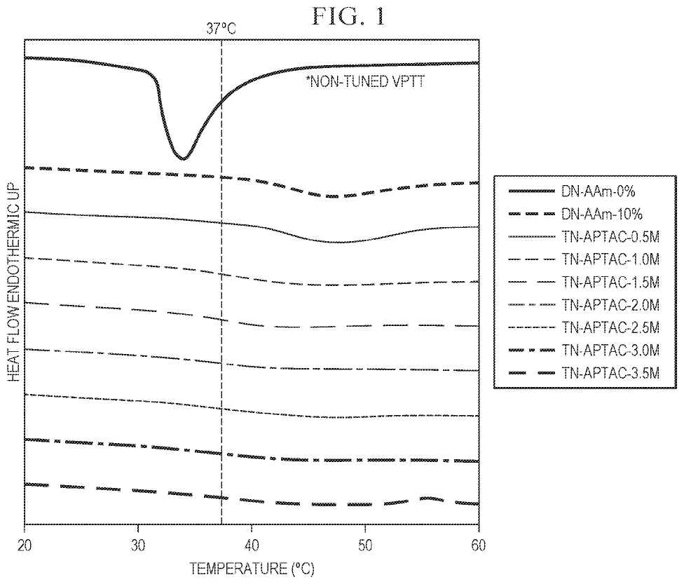

As disclosed herein, TN hydrogels leveraging electrostatic and hydrophobic interactions, which combine asymmetrically crosslinked networks of anionic PAMPS, P (NIPAAm-co-AAm), and cationic poly((3-acylamidopropyl)trimethylammonium chloride) (PAPTAC), are presented. The third network of these hydrogels was controlled to achieve tunable mechanical properties, with an up to ˜3× increase in modulus versus existing DN hydrogels without sacrificing strength, toughness, or water content (FIG. 1). This phenomenon was afforded by reversible, electrostatic crosslinks between the first and third network and hydrophobic interactions within the second network. The unprecedented combination of properties set forth by TN-APTAC hydrogels bolsters the utility of hydrogels as synthetic cartilage grafts, promoting their use in applications requiring an ultra-high modulus.

### Working Examples

Reference will now be made to more specific embodiments of the present disclosure and data that provides support for such embodiments. However, it should be noted that the disclosure below is for illustrative purposes only and is not intended to limit the scope of the claimed subject matter in any way.

TN hydrogels were fabricated using a three-step UV cure process wherein after the first and second cure the hydrogel was placed in a precursor solution for the subsequent network. Hydrogels were denoted TN-X-YM where X is the monomer and Y is the concentration of the third network (e.g., TN-APTAC-1.5M) (Table 2).

TABLE 2
SN, DN, and TN hydrogel network composition details

| | Composition | | | | | | |
|---|---|---|---|---|---|---|---|
| | 1^{st} | 2^{nd} Network⁺ | | | | | |
| | Network* | | AAm(w.r.t. | 3^{rd} Network^{#} | | | |
| Hydrogel | AMPS | NIPAAm | NIPAAm) | APTAC | AAm | AMPS | MEDSAH |
| Single Network | | | | | | | |
| SN-AMPS-1-1.5M | 1.5M | | | | | | |
| Double Network | | | | | | | |
| DN-AAm-0% | 1.5M | 2.0M | 0 wt % | | | | |
| DN-AAm-10% | 1.5M | 2.0M | 10 wt % | | | | |
| Triple Network | | | | | | | |
| TN-APTAC-0.5M | 1.5M | 2.0M | 10 wt % | 0.5M | | | |
| TN-APTAC-1.0M | 1.5M | 2.0M | 10 wt % | 1.0M | | | |
| TN-APTAC-1.5M | 1.5M | 2.0M | 10 wt % | 1.5M | | | |
| TN-APTAC-2.0M | 1.5M | 2.0M | 10 wt % | 2.0M | | | |
| TN-APTAC-2.5M | 1.5M | 2.0M | 10 wt % | 2.5M | | | |
| TN-APTAC-3.0M | 1.5M | 2.0M | 10 wt % | 3.0M | | | |
| TN-APTAC-3.5M | 1.5M | 2.0M | 10 wt % | 3.5M | | | |
| TN-AAm-1.5M | 1.5M | 2.0M | 10 wt % | | 1.5M | | |
| TN-AMPS-1.5M | 1.5M | 2.0M | 10 wt % | | | 1.5M | |
| TN-MEDSAH-1.5M | 1.5M | 2.0M | 10 wt % | | | | 1.5M |

For each hydrogel the first and second network were identical, PAMPS (1.5 M) and P (NIPAAm-co-AAm) (2.0 M NIPAAm, AAm at 10 wt % w.r.t. NIPAAm) respectively, as this combination achieves a high strength and elastic modulus. The inclusion of AAm, a hydrophilic co-monomer to NIPAAm, tuned the VPTT above physiologic range shows dimensional stability (i.e., non-thermoresponsive, no swelling/deswelling) at body temperature (˜37° C.) of the DN hydrogels. It was anticipated that the incorporation of a hydrophilic third network would not interfere with this stability, possibly improving it. Through this addition, the hydrogels were shown to maintain dimensional stability and further tune the VPTT out of physiologic range (FIG. 1). This can be credited to the increased thermal energy barrier to overcome hydrogen bonding as well as the interference of a hydrophilic polymer network penetrating the P (NIPAAm-co-AAm) network.

To assess compatibility with tissue in the body, cytotoxicity of the hydrogels was evaluated. TN-APTAC hydrogels were indirectly exposed to hBMSCs and cultured for 24 hrs before being assessed for secreted lactate dehydrogenase (LDH) due to cell membrane disruption. Poly(ethylene glycol) diacrylate (PEG-DA) hydrogels were simultaneously tested as a well characterized non-cytotoxic control. Comparing the LDH levels of TN-APTAC hydrogels to the PEG-DA control (normalized), no significant difference between compositions was observed (Table 3). Thus, it was confirmed the TN-APTAC hydrogels were cytologically compatible (i.e., non-harming to local cells).

TABLE 3
TN hydrogel detailed relative LDH values (cytocompatibility)

| Hydrogel | Relative LDH Level |
|---|---|
| Control | |
| PEG-DA | 1.00 ± 0.15 |
| Triple Network | |
| TN-APTAC-0.5M | 0.90 ± 0.13 |
| TN-APTAC-1.0M | 0.70 ± 0.18 |
| TN-APTAC-1.5M | 1.21 ± 0.11 |
| TN-APTAC-2.0M | 1.01 ± 0.08 |
| TN-APTAC-2.5M | 0.65 ± 0.16 |
| TN-APTAC-3.0M | 0.81 ± 0.19 |
| TN-APTAC-3.5M | 0.58 ± 0.13 |
| TN-AAm-1.5M | 1.06 ± 0.12 |
| TN-AMPS-1.5M | 1.45 ± 0.20 |
| TN-MEDSAH-1.5M | 1.19 ± 0.15 |

To achieve cartilage-mimetic status, materials not only must match mechanically but hydration levels should be similar as well. Cartilaginous tissues typically have a water content of 60-90% by weight. Commonly, efforts to increase the modulus of hydrogels have been associated with decreasing water contents, especially when attempting to maintain ultra-high strengths due to their inverse relationship. Through the combination of three hydrophilic polymer networks, the TN-APTAC hydrogels obtained water contents around ˜80%, well within the range of cartilaginous tissues such as articular cartilage (65-80%) and IVDs (70-90%) (Table 4).

TABLE 4
SN, DN, and TN hydrogel detailed equilibrium water contents

| Hydrogel | Water Content (%) |
|---|---|
| Single Network | |
| SN-AMPS-1.5M | 96.93 ± 1.11 |
| Double Network | |
| DN-AAm-10% | 85.14 ± 0.61 |
| Triple Network | |
| TN-APTAC-0.5M | 82.09 ± 0.39 |
| TN-APTAC-1.0M | 81.19 ± 2.24 |
| TN-APTAC-1.5M | 78.79 ± 0.33 |
| TN-APTAC-2.0M | 80.01 ± 0.48 |
| TN-APTAC-2.5M | 80.66 ± 0.46 |
| TN-APTAC-3.0M | 79.14 ± 0.72 |
| TN-APTAC-3.5M | 80.30 ± 0.47 |
| TN-AAm-1.5M | 82.17 ± 0.64 |
| TN-AMPS-1.5M | 88.83 ± 0.13 |
| TN-MEDSAH-1.5M | 76.90 ± 0.38 |

These high-water contents are likely to favorably impact the biocompatibility of the hydrogels as well as promote lubrication mechanisms akin to native tissues. Furthermore, the high presence of entrapped and bound water could increase these hydrogel's compressive mechanical properties.

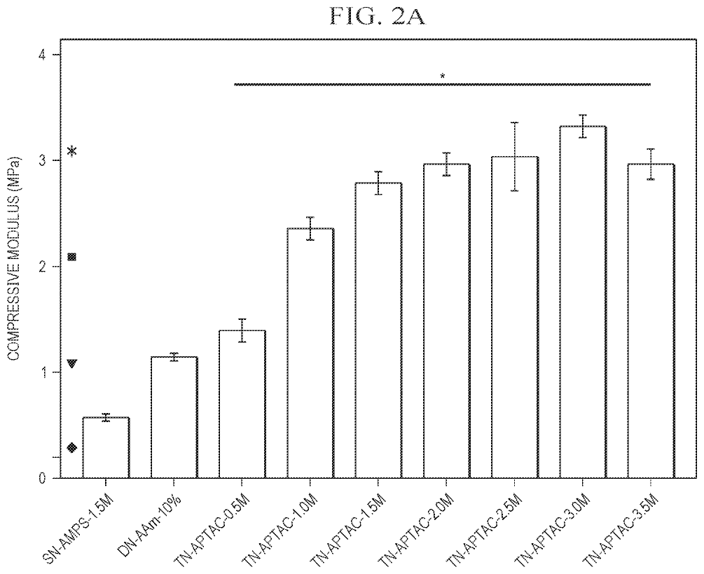

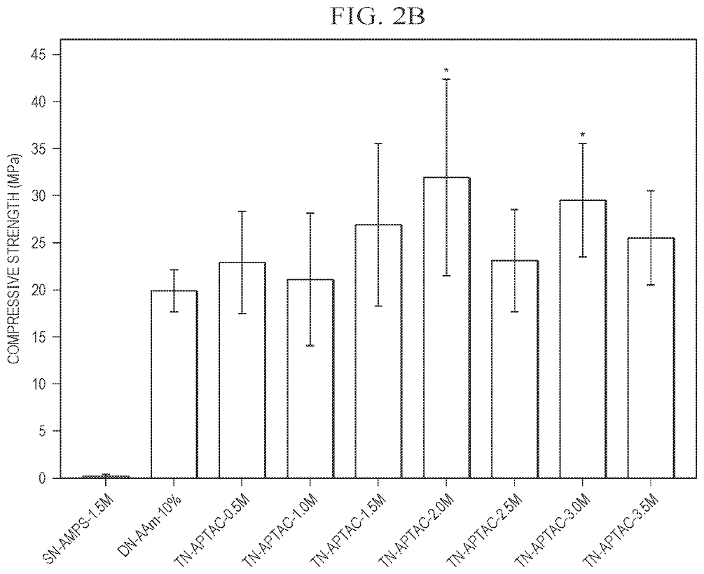

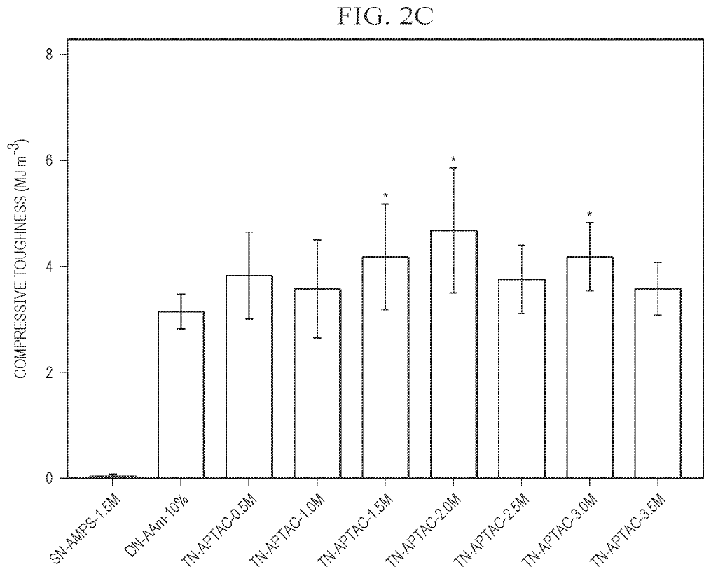

A function of cartilage tissues is load support while allowing for joint articulation. The ability of hydrogels to function in these scenarios has been limited due to their historically low compressive moduli, for example PEG-DA hydrogels commonly have a modulus and strength in the kPa range (˜200 kPa and ˜130 kPa, respectively). Without a modulus mimetic of surrounding tissue, stresses can concentrate on these tissues leading to degeneration or implant failure. Recently, DN hydrogels have emerged as attractive candidates for cartilage replacement due to their ability to obtain a modulus and strength in the MPa range. Complex DN hydrogels composed of PAMPS/P (NIPAAm-co-AAm) wherein hydrophobic interactions of PNIPAAm allow for an apparent increase in crosslinking density—increasing the modulus—have been reported. While these hydrogels had a modulus of ˜1 MPa, articular cartilage has been shown to have depth dependent mechanical properties with the deepest layers reaching a modulus much greater than the surface (˜2.1 MPa). Furthermore, other forms of cartilage such as the temporomandibular joint (TMJ) disc or IVDs have a compressive modulus of ˜2 MPa and 3+ MPa, respectively. Towards increasing the modulus, leverage of electrostatic interactions was investigated. Specifically of interest were internetwork attractions, leading to physical crosslinks between networks that allow for avoidance of brittleness due to their reversible nature. The incorporation of a PAPTAC third network afforded these interactions with the PAMPS first network, leading to a tunable modulus tied to concentration. At very low concentrations (TN-APTAC-0.5 M) only a slight increase in modulus was seen (˜1.4 MPa), indicating low levels of internetwork crosslinks. With rising concentration, the amount of internetwork crosslinks increased resulting in TN-APTAC-1.0M and TN-APTAC-1.5M having moduli of ˜2.4 MPa and ˜2.8 MPa, respectively. A plateau to the increase in modulus was seen to begin around TN-APTAC-2.0M at ˜3 MPa, indicating a maximum influence of internetwork crosslinks, possibly due to saturation (FIGS. 2A-2C; Table 5).

TABLE 5
TN hydrogel detailed compressive mechanical properties

| | Modulus | Strength | Ultimate | Toughness |
|---|---|---|---|---|
| Hydrogel | (MPa) | (MPa) | Strain (%) | (MJ m⁻³) |
| Single Network | | | | |
| SN-AMPS-1.5M | 0.60 ± 0.05 | 0.49 ± 0.11 | 32.39 ± 3.29 | 0.06 ± 0.02 |
| Double Network | | | | |
| DN-AAm-10% | 1.17 ± 0.04 | 20.04 ± 2.15 | 81.06 ± 1.77 | 3.16 ± 0.32 |
| Triple Network | | | | |
| TN-APTAC-0.5M | 1.40 ± 0.10 | 23.09 ± 5.47 | 83.35 ± 3.17 | 3.85 ± 0.85 |
| TN-APTAC-1.0M | 2.39 ± 0.09 | 21.32 ± 6.97 | 85.93 ± 4.68 | 3.62 ± 0.90 |
| TN-APTAC-1.5M | 2.82 ± 0.10 | 27.20 ± 8.65 | 89.72 ± 3.47 | 4.22 ± 1.01 |
| TN-APTAC-2.0M | 2.98 ± 0.09 | 32.15 ± 10.50 | 91.69 ± 3.79 | 4.70 ± 1.18 |
| TN-APTAC-2.5M | 3.06 ± 0.30 | 23.32 ± 5.41 | 90.96 ± 3.94 | 3.79 ± 0.63 |
| TN-APTAC-3.0M | 3.35 ± 0.11 | 29.71 ± 5.99 | 90.93 ± 2.18 | 4.24 ± 0.62 |
| TN-APTAC-3.5M | 2.98 ± 0.15 | 25.63 ± 5.12 | 90.25 ± 2.43 | 3.61 ± 0.51 |
| TN-AAm-1.5M | 1.40 ± 0.08 | 22.48 ± 4.51 | 85.99 ± 2.77 | 3.62 ± 0.66 |
| TN-AMPS-1.5M | 1.16 ± 0.11 | 4.71 ± 1.63 | 71.00 ± 5.92 | 0.93 ± 0.27 |
| TN-MEDSAH-1.5M | 0.96 ± 0.08 | 16.75 ± 4.96 | 85.89 ± 4.03 | 2.75 ± 0.64 |

In concert with internetwork crosslinks, increasing the concentration of APTAC in the third network resulted in higher amounts of crosslinks possibly influencing the modulus as well. In comparison to TN-APTAC hydrogels, polydimethylsiloxane (PDMS) has been shown to exhibit a compressive modulus of ˜2.5 MPa up to ˜3.5 MPa at average and very high crosslinking ratios (10:1 and 5:1, base: curing agent, SYLGARD™ 184), respectively. While attaining such high moduli, the TN-APTAC hydrogels did not become brittle due to the reversible nature of internetwork crosslinks. All compositions reached a strength of at least ˜25 MPa and a toughness of ˜4 MJ m⁻³ (FIGS. 2A-2C; Table 6), highlighting their ability to withstand high load bearing environments (e.g., spine or knee).

TABLE 6
TN-Series detailed instantaneous and final creep strains

| | Instantaneous Strain | Final Strain |
|---|---|---|
| Hydrogel | (t = 0 min, %) | (t = 60 min, %) |
| Double Network | | |
| DN-AAm-10% | 30.81 ± 0.82 | 47.09 ± 1.07 |
| Triple Network | | |
| TN-APTAC-1.5M | 23.28 ± 2.18 | 38.29 ± 3.56 |
| TN-AAm-1.5M | 27.60 ± 0.86 | 40.95 ± 0.11 |
| TN-AMPS-1.5M | 24.88 ± 0.55 | 43.09 ± 0.18 |
| TN-MEDSAH-1.5M | 28.01 ± 0.24 | 42.13 ± 0.48 |

To illustrate the unique effect of PAPTAC to develop the internetwork crosslinks, a series of TN hydrogels with neutral, anionic, and zwitterionic third networks (1.5 M PAAm, PAMPS, and poly(2-(methacryloyloxy)ethyl)dimethyl-(3-sulfopropyl) ammonium hydroxide) (PMEDSAH), respectively) was also evaluated. PAAm would not have internetwork interactions, PAMPS would allow for repulsion, and PMEDSAH could allow for attraction, repulsion, or no interactions. Across these compositions no notable increase in modulus, akin to TN-APTAC-1.5M, from DN-AAm-10% was exhibited (Table 7).

TABLE 7
TN-Series detailed creep strain recovery percent post load removal

| Hydrogel | t = 0 min (%) | t = 1 min (%) | t = 5 min (%) | t = 10 min (%) | t = 30 min (%) |
|---|---|---|---|---|---|
| Double Network | | | | | |
| DN-AAm-10% | 38.52 ± 2.24 | 65.33 ± 5.51 | 72.10 ± 5.55 | 75.17 ± 5.13 | 76.29 ± 4.15 |
| Triple Network | | | | | |
| TN-APTAC-1.5M | 76.95 ± 2.53 | 77.88 ± 2.88 | 78.10 ± 3.02 | 78.13 ± 3.05 | 77.48 ± 2.65 |
| TN-AAm-1.5M | 67.01 ± 1.17 | 83.93 ± 2.98 | 84.94 ± 2.99 | 85.78 ± 2.22 | 86.71 ± 1.30 |
| TN-AMPS-1.5M | 76.22 ± 1.83 | 76.46 ± 1.73 | 76.03 ± 1.58 | 75.47 ± 1.87 | 75.03 ± 1.86 |
| TN-MEDSAH-1.5M | 86.45 ± 0.71 | 87.74 ± 0.62 | 88.35 ± 0.55 | 88.68 ± 0.50 | 89.27 ± 0.52 |

Thus, the increase in modulus by the TN-APTAC hydrogels was determined to be unique due to the presence of internetwork physical crosslinks. Furthermore, investigation into the viscoelastic properties (creep) of the TN hydrogels revealed TN-APTAC-1.5M exhibited a more elastic response than all other TN hydrogels as well as the DN-AAm-10% control. This was exemplified by decreased initial strains and lower final creep strains (post 1 hr constant load) (Table 7). Additionally, TN-APTAC-1.5M achieved its maximum recovery near instantaneously compared to the prolonged recovery of DN-AAm-10% (Table 8).

TABLE 8
TN-APTAC detailed tensile mechanical properties

| | Modulus | Strength | Ultimate | Toughness |
|---|---|---|---|---|
| Hydrogel | (MPa) | (MPa) | Strain (%) | (MJ m⁻³) |
| Double Network | | | | |
| DN-AAm-10% | 1.02 ± 0.04 | 1.39 ± 0.10 | 117.2 ± 23.9 | 1.00 ± 0.31 |
| Triple Network | | | | |
| TN-APTAC-0.5M | 1.12 ± 0.08 | 1.55 ± 0.09 | 109.9 ± 20.8 | 1.02 ± 0.33 |
| TN-APTAC-1.0M | 1.64 ± 0.44 | 1.51 ± 0.15 | 118.9 ± 16.3 | 1.19 ± 0.26 |
| TN-APTAC-1.5M | 2.57 ± 0.14 | 1.20 ± 0.04 | 100.6 ± 14.2 | 0.85 ± 0.16 |
| TN-APTAC-2.0M | 2.93 ± 0.09 | 1.11 ± 0.03 | 106.0 ± 8.1 | 0.89 ± 0.11 |
| TN-APTAC-2.5M | 2.86 ± 0.09 | 1.10 ± 0.04 | 115.6 ± 25.2 | 0.98 ± 0.28 |
| TN-APTAC-3.0M | 3.05 ± 0.10 | 1.06 ± 0.03 | 129.1 ± 24.7 | 1.08 ± 0.23 |
| TN-APTAC-3.5M | 3.00 ± 0.15 | 1.03 ± 0.06 | 115.6 ± 11.9 | 0.92 ± 0.12 |

These phenomena were likely caused by the increased crosslinking density of TN-APTAC hydrogels limiting chain restructuring due to relaxation.

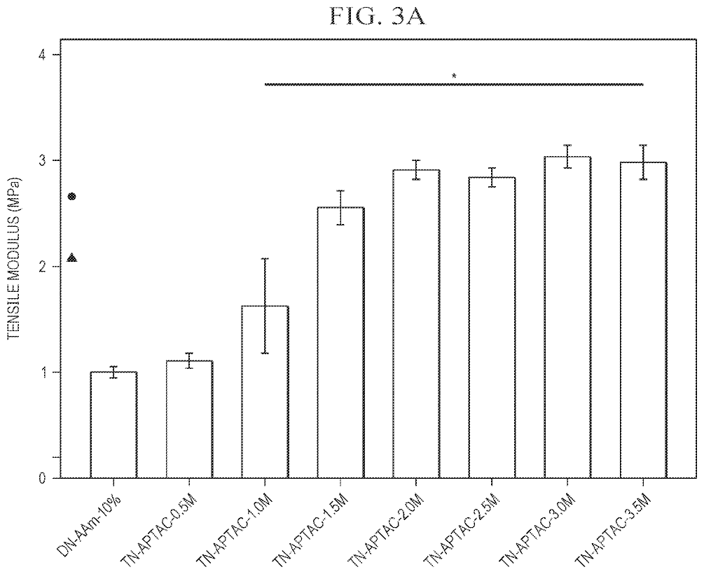

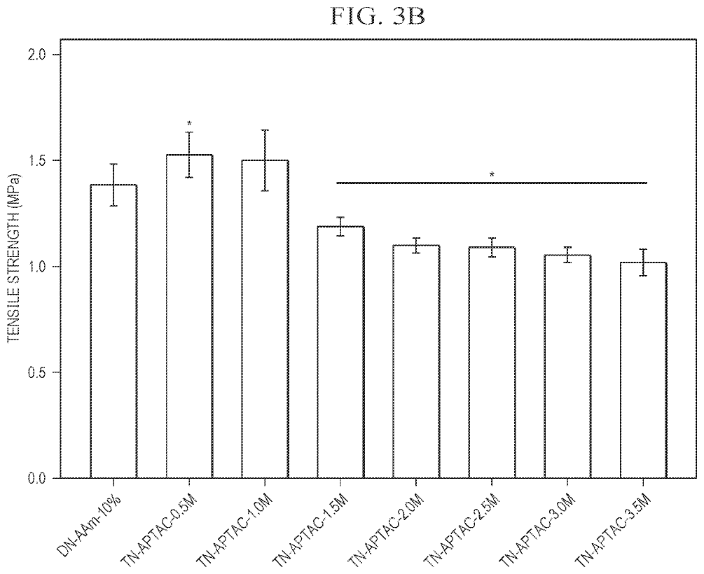

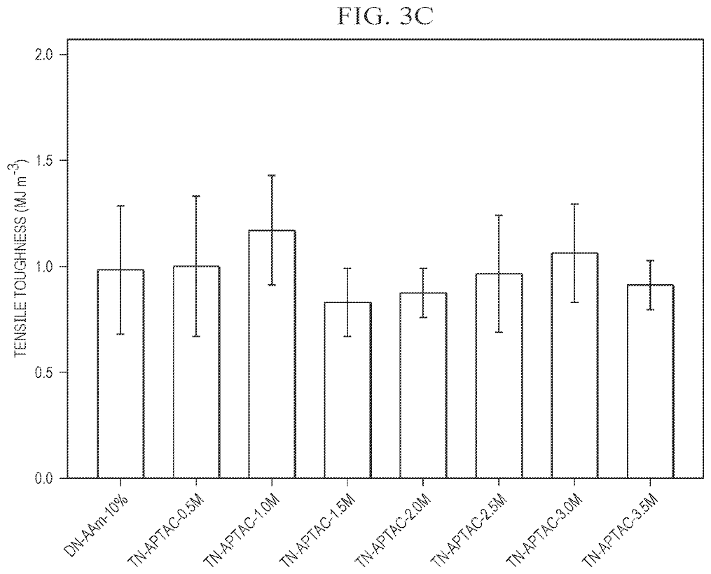

While compressive loads represent a primary function of cartilage tissues, tensile stresses are common is scenarios such as flexion/extension of IVDs (annulus fibrosus), respiratory function (costal cartilage), and compressive loading resulting in circumferential tensile stresses (e.g., annulus fibrosus or meniscus). Conventional hydrogels typically lack the necessary tensile properties to function in these environments and fiber reinforced hydrogels often suffer from low water contents. Similar to compression, internetwork crosslinks could increase the modulus of hydrogels in tension without decreasing water content. Evaluation of the TN-APTAC hydrogels saw trends akin to compression, tunable moduli with increasing APTAC concentration. Transitionary increases were seen for TN-APTAC-0.5M to TN-APTAC-1.5M with moduli ranging from ˜1 MPa to ˜2.5 MPa, respectively. This was followed by a plateau at ˜3 MPa for TN-APTAC-2.0M onwards, exceeding that of the circumferential annulus fibrosus toe modulus (˜2+ MPa) and reaching the lower end of tracheal cartilage (FIGS. 3A-3C; Table 8). Comparatively, polyurethane elastomers under physiologic conditions have been shown to possess a modulus of ˜4.5 MPa, with the potential to decrease depending on composition. Not only were moduli attained in the MPa range, strengths exhibited by all TN-APTAC hydrogels were in the MPa range (˜1 MPa). This was accompanied with high strains at failure (˜100%) and corresponding toughnesses of ˜1 MJ m⁻³ (FIGS. 3A-3C; Table 8). Together these represent an unmatched combination of tensile properties for hydrogels, approaching levels of non-hydrated polymeric materials and highlighting the ability to create synthetic materials for replacement of tissues such as tracheal cartilage.

The work presented in this application has explored the development of PAMPS/P (NIPAAm-co-AAm)/PAPTAC TN hydrogels and their suitability as synthetic cartilage replacements. Hydrogels have historically been limited in use as synthetic replacements for load bearing applications due to their low moduli. As shown herein, electrostatic interactions-forming physical internetwork crosslinks-were explored as a way to increase the modulus without diminishing strength and water content. These interactions enabled TN-APTAC hydrogels to have a tunable modulus tied to the concentration of PAPTAC in the third network. Increasing concentrations allowed for a higher amounts of internetwork crosslinks, leading to higher moduli with a plateau seen at ˜3 MPa (compression and tension). This increase in modulus was not associated with increased brittleness of the hydrogels due to the reversible nature of physical crosslinks. Furthermore, through the incorporation of a hydrophilic third network a high, cartilage-mimetic water content was reached. It was also confirmed that TN-APTAC hydrogels were cytocompatible through culture with hBMSCs and dimensionally stable by tuning PNIPAAm's VPTT beyond physiologic range. Through the achievement of an unprecedented set of cartilage-mimetic mechanical and hydration properties, TN-APTAC hydrogels bolster the utility of hydrogels to serve as synthetic grafts for IVDs, TMJ discs, and tracheal cartilage, among other applications.

Reference will now be made to particular materials and methods utilized by various embodiments of the present disclosure. However, it should be noted that the materials and methods presented below is for illustrative purposes only and is not intended to limit the scope of the claimed subject matter in any way.

Materials. Acrylamide (AAm, >99%), 2-acrylamido-2-methylpropane sulfonic acid (AMPS, 97%), (3-acrylamidopropyl)trimethylammonium chloride solution (APTAC, 75 wt % in H₂O), N,N′-methylenebisacrylamide crosslinker (BIS, 99%), N-isopropylacrylamide (NIPAAm, 97%), [2-(methacryloyloxy)ethyl]dimethyl-(3-sulfopropyl) ammonium hydroxide (MEDSAH, 95%), 2-oxoglutaric acid (2-oxo, 99.0-101.0%), triethylamine, acryloyl chloride, Irgacure 2959, ethanol (EtOH), and diethyl ether were obtained from Millipore-Sigma. Linear poly(ethylene glycol) (PEG, 8 kDa) was purchased from Fluka BioChemika. Dichloromethane (DCM), potassium carbonate (K₂CO₃), and magnesium sulfate (MgSO₄) were obtained from Fisher. For hydrogel fabrication, deionized (DI) water (18 MΩ2·cm, Cascada LS MK2, Pall) was used. Minimum Essential Medium (α-MEM), heat-inactivated fetal bovine serum (FBS), GlutaMAX, and antibiotic solution (penicillin (10,00 IU mL⁻¹) and streptomycin (10,000 μg mL⁻¹)) were obtained from Gibco. Dulbecco's phosphate buffered saline (DPBS) was obtained from Corning. For cytotoxicity testing a lactate dehydrogenase (LDH) detection kit was purchased for Roche. Human bone marrow derived mesenchymal stems cells (hBMSCs) were obtained from one donor (Texas A&M Institute for Regenerative Medicine).

Triple Network Hydrogel Fabrication. Triple Network (TN) hydrogels were fabricated in a multi-step process where each network was UV cured. Single network (SN) hydrogels were cured and subsequently soaked in a double network (DN) precursor solution which was then cured, followed by a similar process for the third network. The SN precursor solution was composed of AMPS (1.5 M), BIS crosslinker (4 mol % w.r.t. AMPS), and 2-oxo photo initiator (0.1 mol % w.r.t. AMPS) in DI water. This solution was UV cured (UV-transilluminator, 6 mW cm⁻², 365 nm) in a glass slide mold (˜1 mm spacers) for 5 hours, flipping the mold every 15 min for the first hour and every hour for the remaining 4 hours to maintain symmetry. Post curing, SN hydrogels were soaked in a DN precursor solution for 48 hrs. The DN precursor solution was composed of NIPAAm (2.0 M), AAm (10 wt % w.r.t. NIPAAm), BIS (0.1 mol % w.r.t. NIPAAm), and 2-oxo (0.1 mol % w.r.t. NIPAAm) in DI water. After soaking, the hydrogel was UV cured in a glass slide mold (˜1.25 mm spacers) for 5 hours in an ice bath (˜7° C.) following a similar flipping pattern to curing of SN hydrogels. Post curing, DN hydrogels were soaked in a TN precursor solution for 48 hrs. The TN precursor solution was composed of monomer, BIS (0.1 mol % w.r.t. monomer), and 2-oxo (0.1 mol % w.r.t. monomer) in DI water. The monomer was either APTAC (0.5-3.5 M), AAm (1.5 M), AMPS (1.5 M), or MEDSAH (1.5 M). After soaking, the hydrogel was UV cured in a glass slide mold (˜1.25 mm spacers) for 5 hours in an ice bath (˜7° C.) following a similar flipping pattern to curing of SN and DN hydrogels. Post curing, TN hydrogels were soaked in DI water for at least 1 week before testing. TN hydrogels were denoted TN-X-YM where X is the monomer and Y is the concentration (e.g., TN-APTAC-1.5M) (FIG. 1; Table 2).

Double Network Hydrogel Fabrication. DN hydrogels (control) were fabricated using a similar process, differing post curing of the first network. SN hydrogels were soaked in a DN precursor composed of composed of NIPAAm (2.0 M), AAm (0 wt % or 10 wt % w.r.t. NIPAAm), BIS (0.1 mol % w.r.t. NIPAAm), and 2-oxo (0.1 mol % w.r.t. NIPAAm) in DI water. After soaking, the hydrogel was UV cured in a glass slide mold (˜1.25 mm spacers) for 5 hours in an ice bath (˜7° C.) following a similar flipping pattern to curing of SN hydrogels. Post curing, DN hydrogels were soaked in DI water for at least 1 week before testing. DN hydrogels were denoted DN-AAm-X % where X is the weight percent of AAm w.r.t. NIPAAm (e.g., DN-AAm-10%) (Table 2).

Volume Phase Transition Temperature (VPTT). The VPTT of hydrated TN hydrogels was evaluated through differential scanning calorimetry (DSC, TA Instruments Q100). A ˜10 mg sample was cut and blotted dry with a Kim Wipe before being inserted and sealed into a hermetic pan. The sample was equilibrated to (° C.), then two continuous cycles of heating to 65° C. and back to 0° C. at 3° C. min-1 were completed. The VPTT was characterized by the peak temperature of the endotherm (Tₘₐₓ) and the initial temperature at the start of the endothermic phase transition (To). Data reported are from the second heating cycle to avoid thermal history and simulate an arbitrary n^{th} heating cycle.

Cytocompatibility. Human bone marrow derived mesenchymal stem cells (hBMSCs) (Texas A&M Institute for Regenerative Medicine) from one donor were cultured. Briefly, the cells were maintained in Minimum Essential Medium-α (α-MEM, Gibco) supplemented with 10% MSC-qualified, heat-inactivated fetal bovine serum (FBS, Gibco) and 1× GlutaMAX (Gibco) in a 37° C.-5% CO₂ jacketed incubator. Twenty-four hours prior to seeding in 24-well plates for experimentation, 1% antibiotic solution (10,000 IU mL⁻¹ penicillin, 10,000 μg mL⁻¹ streptomycin) was added to the growth medium, serving as the experimental medium.

TN-hydrogels were fabricated as described and were sterilized via soaking in 100% ethanol (EtOH) followed by soaks in mixtures of EtOH and Dulbecco's phosphate buffered saline (DPBS) (75:25%, 50:50%, 25:75%, and 0:100%, respectively) each for 20 min. PEG-DA (8 kDa) hydrogels known for their cytocompatibility were synthesized to be used as a non-cytotoxic control. 1% v/v photoinitiator (262 mg/mL Irgacure 2959 in 70% EtOH) was added to 10% w/v PEG-DA polymer precursor solution (in DPBS) and passed through a 0.22 μm filter for sterilization. Volumes equivalent to those of the TN-hydrogels were then dispensed into the wells of a 48-well plate and exposed to longwave UV light (˜10 mW/cm², 6 mins). Following, TN and PEG-DA hydrogels were rinsed four times for 5 minutes in fresh DPBS before being transferred into the experimental medium for 24 hours prior to assessing cytocompatibility.

hBMSCs were seeded into 24-well plates at a density of 10,000 cells/cm² and allowed to adhere for 30 min at 37° C.-5% CO₂. The hydrogels were then transferred into transwell inserts then placed on top of seed hBMSCs which were then cultured for 24 hours before cell culture supernatant was extracted for cytotoxicity assessment. Cytotoxicity of cellular exposure to the TN-hydrogels was assessed through measurement of secreted lactate dehydrogenase (LDH) using an LDH detection kit (Roche). To quantitatively assess the number of damaged cells contributing LDH, a standard ranging from 100,000 cells/mL to 0 cells/mL was created using a serial dilution of lysed hBMSCs prepared immediately before the assay.

Equilibrium Water Content (EWC). EWC of the hydrogels was determined by drying a swollen hydrogel or cartilage sample (6 mmØ×˜2 mm thickness) in an oven overnight at 60° C. under high vacuum (30 in. Hg). Water content was calculated after drying as

$$\frac{{W}_{s} - {W}_{d}}{{W}_{s}} \times 100 ,$$

where Wₛ and W_{d} were the swollen and dry weight, respectively.

Unconfined Compression. Compressive mechanical properties (e.g., elastic modulus, strength) were evaluated at room temperature with an Instron 5944. Hydrogel samples were punched into discs (6 mm Ø×˜2 mm thickness) using a biopsy punch. Samples were blotted with a Kim Wipe to remove surface water. An initial preload of 0.2 N was applied to each sample and subsequently the strain was zeroed. Samples were compressed at a rate of 1 mm min-1 until fracture. The elastic modulus was calculated using the linear region (0-10%) of the stress-strain curve. The compressive strength was defined as the stress at the point of fracture. Accordingly, the ultimate strain was the strain value at fracture. Toughness was determined by integration of the stress-strain curve to the point of fracture.

Creep. Creep was analyzed using dynamic mechanical analysis (DMA, TA Instruments Q800) and the viscoelastic behavior of the hydrogels was compared. Samples were prepared identically to unconfined compression. A 0.35 MPa load was applied to samples for 1 hr, followed by removal of the load and a 30 min recovery period. Instantaneous and final creep strain were analyzed as well as strain recovery immediately and at 1, 5, 10, and 30 min post load removal.

Tension. Tensile mechanical properties (e.g., modulus, strength) were evaluated at room temperature with an Instron 5944. Hydrogel samples were punched into dog-bone samples using a certified punch (ASTM D1708-18). Samples were blotted with a Kim Wipe to remove surface water. Samples were displaced at a rate of 10 mm min-1 until fracture. The elastic modulus was calculated using the linear region (0-10%) of the stress-strain curve. The compressive strength was defined as the stress at the point of fracture. Accordingly, the ultimate strain was the strain value at fracture. Toughness was determined by integration of the stress-strain curve to the point of fracture.

Statistics. For unconfined compression, tension, EWC, and cytocompatibility statistical analyses were completed using a one-way analysis of variance (ANOVA) with Dunnet's multiple comparisons test. Creep recovery was analyzed using a two-way ANOVA with Tukey's multiple comparisons test. Analyses were conducted with GraphPad Prism (Version 9.2.0) using a standard alpha level of 0.05. All comparisons with p<0.05 were considered statistically significant.

In summary, multi-network hydrogels composed of three networks or more for use as cartilage substitutes for the various cartilage types that span the body (e.g., articular, tracheal, coastal, intervertebral disc, etc.) has been disclosed. Previously, double network hydrogels that mimic the mechanical properties of articular caltilage, including a modulus in the MPa range have been developed. These were composed of asymmetrically crosslinked networks of PAMPS and NIPAAm copolymerized with acrylamide AAm (PAMPS/P [NIPAAm-co-AAm]). Superior mechanical properties of these hydrogels were afforded by the incorporation of NIPAAm in the second network, which allowed for an apparent increase in crosslinking density through reversible hydrophobic interactions. This allowed for the double network hydrogels to reach strengths of ˜20 MPa and elastic moduli of ˜1 MPa while maintaining a cartilage like water content (˜84%). Further study of these PNIPAAm double network hydrogels illustrated their ability to mimic articular cartilage's tribological properties. In a synovial fluid simulant, mixed-boundary lubrication was suppressed and double network hydrogels were pushed to similar elasto-hydrodynamic regimes as articular cartilage. While these PNIPAAm double network hydrogels show promise towards serving as a resurfacing material for articular cartilage, their modulus does not extend into the range of other cartilaginous tissues (e.g., IYO or tracheal cartilage).

Herein, triple network and other multi-network hydrogels leveraging electrostatic and hydrophobic interactions, which combine asymmetrically cross linked networks of anionic PA MPS, P (NIPAAm-co-AAm), and cationic poly((3-acylamidopropyl)trimethylammonium chloride) (PAPTAC), are described. The third network of these hydrogels was controlled to achieve tunable mechanical properties, with an up to ˜3× increase in modulus versus existing double network hydrogels without sacrificing strength, toughness, or water content. This phenomenon was afforded by reversible, electrostatic crosslinks between the first and third network and hydrophobic interactions within the second network. The unprecedented combination of properties set forth by TN-APTAC hydrogels bolsters the utility of hydrogels as synthetic cartilage grafts, promoting their use in applications requiring an ultra-high modulus.

Furthermore, these cartilage mimetic hydrogels can optionally be used in conjunction with another material, such as a regenerative scaffold (e.g., hydrogel and non-hydrogels) or a metal (e.g., titanium, stainless steel), serves as an anchoring device or “base”. To further enhance tissue integration with surrounding cartilage, the double network hydrogels may be porated either throughout the material or solely around the perimeter nearest the tissue interface. Alternatively, or in conjunction with poration, a polydopamine coating may be applied to all or part of the cartilage mimetic gel to promote tissue adhesion and integration. Various fillers and biologics may also be incorporated to enhance various properties.

Triple network hydrogels fabricated in a multi-step process where each network was UV cured. Typically, a single network hydrogel is cured and subsequently soaked in a double network precursor solution which is then cured, followed by a similar process for the third network. Networks maybe asymmetrically crosslinked such that the relative crosslink density (i.e., “highly” vs. “lightly”) varies among the networks. Electrostatic forces and hydrophobic interactions are introduced by controlling network chemistry

In one example, a triple network series is as follows: The single network precursor solution was composed of AMPS (1.5 M), BIS crosslinker (4 mol % w.r.t. AMPS), and 2-oxo photo initiator (0.1 mol % w.r.t. AMPS) in DI water. This solution was UV cured (UV-transilluminator, 6 mW cm-2, 365 nm) in a glass slide mold (˜1 mm spacers) for 5 hours, flipping the mold every 15 min for the first hour and every hour for the remaining 4 hours to maintain symmetry. Post curing, single network hydrogels were soaked in a double network precursor solution for 48 hrs. The double network precursor solution was composed of NIPAAm (2.0 M), AAm (10 wt % w.r.t. NIPAAm), BIS (0.1 mol % w.r.t. NIPAAm), and 2-oxo (0.1 mol % w.r.t. NIPAAm) in DI water. After soaking, the hydrogel was UV cured in a glass slide mold (˜1.25 mm spacers) for 5 hours in an ice bath (˜7° C.) following a similar flipping pattern to curing of single network hydrogels. Post curing, double network hydrogels were soaked in a triple network precursor solution for 48 hrs. The triple network precursor solution was composed of monomer, BIS (0.1 mol % w.r.t. monomer), and 2-oxo (0.1 mol % w.r.t. monomer) in DI water. The monomer was either APTAC (0.5-3.5 M), AAm (1.5 M), AMPS (1.5 M), or MEDSAH (1.5 M). After soaking, the hydrogel was UV cured in a glass slide mold (˜1.25 mm spacers) for 5 hours in an ice bath (˜7° C.) following a similar flipping pattern to curing of single network and double network hydrogels. Example materials include: AMPS: poly(2-acrylamido-2-methylpropane sulfonic acid (monomer), BIS: N,N′-methylenebisacrylamide (crosslinker), 2-oxoglutaric acid (UV-initiator), DI deionized water (solvent), NIPAAM: N-isopropylacrylamide (monomer), AAm: Acrylamide (AAm, monomer), MEDSAH: 2-(methacryloyloxy)ethyl]dimethyl-(3-sulfopropyl) ammonium hydroxide (monomer), and APTAC: (3-acrylamidopropyl)trimethylammonium chloride solution (monomer).

To porate the cartilage mimetic hydrogels (at the tissue-contacting perimeter), a porogen (e.g., thermoplastic poragen) is added during the first UV-cure step to all or part of the mold which remains present until the triple network or multi-network hydrogel is fully formed. Interconnectivity of pores can optionally be achieved through annealing to lightly fuse the porogens together. Then, the porogens are removed from the hydrogels via soaking in a solvent to induce dissolution, producing pores with tunable size (e.g., ˜100 microns) and potential interconnectivity to promote adjacent tissue integration into the hydrogel.

To coat the hydrogel with polydopamine, cither all or part of the hydrogel is submerged within a dopamine solution (e.g., dopamine hydrochloride for 24 hr). If used in conjunction with poration, this coating is applied after the porogens have been removed.

The multi-network hydrogels of the present disclosure have high tunability of size and geometry, no post-processing of the materials is necessary, no harsh chemicals needed for synthesis or as curing agents, no required heat for curing which can negatively affect thermosensitive materials, tunable pore size and interconnectivity through selected porogens, and have a simple coating technique that can be applied directly to the hydrogel material

Triple or multinetwork hydrogels based on NIPAAm and electrostatic monomers achieve an unprecedented combination of cartilage-like (for different cartilage types) moduli (i.e., stiffness), and hydration. These results are afforded by reversible, electrostatic crosslinks and hydrophobic interactions.

Non-Thermoresponsive hydrogels. The addition of a neutral, hydrophilic comonomer (e.g., AAm) into the NIPAAm network allowed for tunability of the volume phase transition temperature (VPTT). AAm has been used to adjust the VPTT of NIPAAm single network hydrogels, but have not previously been used for this reason in triple network hydrogels. By raising the VPTT above physiologic range, these hydrogels could exhibit mechanical stability within the body for use as non-thermosensitive implants.

Perimeter poration. The use of a porogen within a hydrogel to spatially control pore size and interconnectivity is a significant enhancement towards ultra-strong hydrogels capable of integration with the surrounding tissue. Most double network hydrogels are non-porous, with mesh sizes in the 1-10's nm range that does not allow for the migration of cells into the material.

Polydopamine coating. The use of polydopamine as a tissue adhesion promoter has shown promise. This coating has not been applied to hydrogels, making this a unique combination of both replacement and regenerative approaches to achieve successful tissue integration.

Connection with an anchoring base. In the case of resurfacing of articular joints, the combination of a cartilage replacement material in conjunction with an anchoring base is important for long term stability. Examples of potential “bases” are regenerative, polymeric scaffolds and metal/alloys. Notably, porous regenerative scaffolds can regenerate the underlying bone while the cartilage mimetic hydrogel immediately supports mechanical loads after implantation. Alternatively, the use of a titanium pin as a base has been used in resurfacing implants. By utilizing this established anchoring pin with the cartilage mimetic hydrogel herein, a closer match the strength, modulus, and hydration of cartilage can be achieved compared to current resurfacing techniques. Additionally, triple network or multinetwork hydrogels may be combined to develop devices with regionally-specific properties.

A myriad of cartilage types span the human body, performing critical functions that necessitate high moduli (˜1 10 MPa, compressive). When these tissues are lost or damaged, repair is limited due to the low healing capacities associated with avascularity. Clinical repair strategies are primarily focused on auto- or allografting procedures, including osteochondral autograft transfer (OATS) for articular cartilage, and laryngotracheal reconstruction with harvested coastal cartilage. Nonetheless, these grafting treatments commonly suffer from donor site morbidity, graft availability, fibrocartilage formation, and graft resorption. To overcome these limitations, synthetic replacements are of significant interest; several have emerged (e.g., artificial intervertebral discs and articular cartilage focal resurfacing implants). The loading that must be endured has prompted the use of synthetic materials whose moduli greatly exceeds that of native cartilage types. However, this resulting mechanical mismatch is a primary source of failure, wherein altering mechanical stimuli of adjacent cartilage or bone leads to its deterioration. Moreover, modulus mismatch is associated with poor integration of the replacement material with surrounding native cartilage, arising from unequal strain responses to applied load. Joint functionality is also highly dependent on modulus matching (e.g., preservation of disc height while replicating motion segment mechanics). Native cartilage is characterized by significant water content, as resulting osmotic forces give rise to their characteristic moduli and viscoelastic properties. The unprecedented combination of properties set forth by TN-APTAC hydrogels bolsters the utility of hydrogels as synthetic cartilage grafts, promoting their use in applications requiring an ultra-high modulus. Therefore, cartilage substitutes are ideally based on synthetic materials that not only achieve similar moduli, but also do so with similar hydration.

In summary, these triple network hydrogels or multi-network hydrogels can be used as synthetic grafts in the above indications (e.g., articular cartilage resurfacing, laryngotracheal reconstruction, intervertebral disc replacement, etc.). Fabrication affords ease to produce specimens with custom size and geometry.

Currently various procedures for articular cartilage resurfacing exist. (1) Osteochondral Autograft Transfer System (OATS): In this procedure, healthy tissue is harvested from undamaged regions of the knee in the form of cylindrical autografts and transferred to predrilled sites in the defect area. (2) Microfracture: To promote healing of the cartilage defect, microfractures are made in the exposed bone to release bone marrow stem cells (BMSCs) to facilitate chondrogenesis. (3) Autologous Chondrocyte Implantation (ACI): ACI is a recently approved alternative method to promote healing of articular cartilage defects that requires a two-stage procedure. First, chondrocytes are harvested from the patient and expanded for 6-8 weeks to produce a large enough quantity of the cells for implantation. Second, a small patch is sewn over the articular cartilage defect and the expanded chondrocytes are injected underneath to regenerate the native hyaline cartilage. (4) Focal Knee Resurfacing: A synthetic autograft alternative has been developed, although, it has not obtained IDE approval in the United States. This focal knee resurfacing device is generally an ultra-high molecular weight polyethylene (UHMWPE) capped titanium (Ti) screw that can be implanted in the defect site to replace the damaged cartilage. (5) Total Knee Replacements (TKRs): If none of the aforementioned procedures are available for the patient, a TKR is necessary. This surgery involves the complete removal of the injured knee joint and replacement with a prosthetic knee, most commonly composed of a Ti stem and an UHMWPE articulating surface.

In view of the procedures, benefits of a synthetic cartilage hydrogel can include, without limitation, avoidance of donor site morbity, currently neonates and pediatric populations have limited supply of cartilage tissue for autografting (synthetic cartilage hydrogels can be used as an alternative), eliminations on limits of defect size, avoidance of long and multi-step procedures, achievement of more similar properties to native cartilage, and providing less expensive and longer-lasting options.

Although various embodiments of the present disclosure have been illustrated in the accompanying Drawings and described in the foregoing Detailed Description, it will be understood that the present disclosure is not limited to the embodiments disclosed herein, but is capable of numerous rearrangements, modifications, and substitutions without departing from the spirit of the disclosure as set forth herein.

The term “substantially” is defined as largely but not necessarily wholly what is specified, as understood by a person of ordinary skill in the art. In any disclosed embodiment, the terms “substantially”, “approximately”, “generally”, and “about” may be substituted with “within [a percentage] of” what is specified, where the percentage includes 0.1, 1, 5, and 10 percent.

The foregoing outlines features of several embodiments so that those skilled in the art may better understand the aspects of the disclosure. Those skilled in the art should appreciate that they May readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure, and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a”, “an”, and other singular terms are intended to include the plural forms thereof unless specifically excluded.

1. A multi-network hydrogel comprising:
a first network, wherein the first network comprises an anionic first network;
a second network, wherein the second network comprises a neutral second network; and
a third network, wherein the third network comprises a cationic third network.

2. (canceled)

3. The multi-network hydrogel of claim 1, wherein the first network comprises an anionic poly(2-acrylamido-2-methylpropane sulfonic acid) (PAMPS) first network.

4. The multi-network hydrogel of claim 1, wherein the second network comprises a neutral monomer copolymerized with acrylamide (AAm).

5. The multi-network hydrogel of claim 4, wherein the neutral monomer comprises N-isopropylacrylamide (NIPAAm).

6. The multi-network hydrogel of claim 1, wherein the second network comprises a neutral P (NIPAAm-co-AAm) second network.

7. The multi-network hydrogel of claim 1, wherein the third network comprises a cationic poly((3-acylamidopropyl)trimethylammonium chloride) (PAPTAC) third network.

8. The multi-network hydrogel of claim 1, wherein the first network, the second network, and the third network form a PAMPS/P (NIPAAm-co-AAm)/PAPTAC triple network hydrogel.

9. The multi-network hydrogel of claim 1, comprising a water content in a range between 70 to 90%.

10. The multi-network hydrogel of claim 1, comprising a modulus in a range between 0.90 and 3.5 MPa.

11. The multi-network hydrogel of claim 1, comprising a strength in a range between 4.5 and 35 MPa.

12-13. (canceled)

14. The multi-network hydrogel of claim 1, further comprising pores.

15. The multi-network hydrogel of claim 14, wherein the first network comprises a porogen in a precursor used to form the first network, and wherein pores were formed via curing of the precursor and subsequent dissolution of the porogen.

16. (canceled)

17. The multi-network hydrogel of claim 14, wherein the pores have a size in a range between 75 to 125 microns.

18. The multi-network hydrogel of claim 1, further comprising a polydopamine coating.

19. The multi-network hydrogel of claim 1, further comprising an anchoring base.

20. The multi-network hydrogel of claim 19, wherein the anchoring base at least one of a regenerative polymer scaffold, titanium pins, metals, or metal alloys.

21-22. (canceled)

23. A method of forming the multi-network hydrogel of claim 1, the method comprising:
curing a first layer precursor solution via ultraviolet (UV) light thereby forming a single network hydrogel;
soaking the single network hydrogel in a second layer precursor solution;
curing the single network hydrogel via UV light thereby forming a double network hydrogel;
soaking the double network hydrogel in a third layer precursor solution; and
curing the double network hydrogel via UV light thereby forming the multi-network hydrogel.

24. The method of claim 23, wherein the first layer precursor solution comprises a porogen.

25. The method of claim 24, further comprising:
dissolving the porogen, subsequent to curing the first layer precursor solution; and
forming pores as a result of dissolution of the porogen from the single network hydrogel.

26. The method of claim 23, further comprising doping at least a portion of the multi-network hydrogel with a dopamine solution.
